(19) United States
(12) Patent Application Publication
(10) Pub. No.: US 2008/0051574 A1
(43) Pub. Date: Feb. 28, 2008

(54) Control of Ph By Direct Addition of Carbonates and Bicarbonates During Concentration of Organics Solvent Extracts of 6-Acetyl-4,1',6' Trichlorogalactosucrose and 4,1',6' trichlorogalactosucrose

(76) Inventors: Rakesh Ratnam, Maharashtra (IN); Sundeep Aurora, Maharashtra (IN)

Correspondence Address: NATH & ASSOCIATES, 112 South West Street, Alexandria, VA, 22314, US

(21) Appl. No.: 11/792,702

(22) PCT Filed: Dec. 9, 2005
(86) PCT No.: PCT/IN05/00407

§ 371 Date: Jun. 8, 2007

(30) Foreign Application Priority Data
Dec. 10, 2004 (IN) 1315/MUM/2004

(51) Int. Cl. C07H 1/00

(52) U.S. Cl. 536/126

(57) ABSTRACT

A novel process is described for control of pH where acid neutralizing agents, including carbonates and bicarbonates of metals and alkaline earth metals, are used in solid form in process of large scale manufacture of a chlorinated sucrose, particularly 4,1′, 6′ trichlorogalactosucrose (TGS) to neutralize acidity formed when ester group containing organic solvent solutions of TGS or 6-acetyl-TGS are concentrated on a large scale. This novel method of pH control is applicable to all organic synthesis reactions where acid neutralization needs to be achieved in as much non-aqueous condition as possible. Also is described a process where use of MTBE could be used for extracting or dissolving 6-acetyl-TGS or TGS instead of ester containing organic solvents which can be concentrated without the need of pH control.

### TECHNICAL FIELD

The present invention relates to a process and a novel strategy for synthesis of chlorinated sucrose, 1′-6′-Dichloro-1′-6′-DIDEOXY-β-Fructofuranasyl-4-chloro-4-deoxy-galactopyranoside.

### BACKGROUND OF THE INVENTION

Chlorinated sucrose preparation is a challenging process due to the need of chlorination in selective less reactive positions in sucrose molecule in competition with more reactive positions. Generally, this objective is achieved by a procedure which involves essentially protecting the primary hydroxy group in the pyranose ring of sugar molecule by converting it to either aromatic or aliphatic esters or Orthoesters, and the protected sucrose is then chlorinated in the desired positions (1′-6′ & 4) to give the acetyl derivative of the product, which is then deacylated to give the desired product 1′-6′-Dichloro-1′-6′-DIDEOXY-β-Fructofuranasyl-4-chloro-4-deoxy-galactopyranoside i.e. 4,1′,6′ trichlorogalactosucrose (TGS).

Strategies of prior art methods of production of TGS are based on following: Sucrose-6-acetate is chlorinated by Vilsmeier-Haack reagent to form 6-acetyl-4,1′, 6′trichlorogalactosucrose (6-acetyl-TGS). After chlorination, the deacetylation of 6-acetyl-TGS to TGS is carried out in the reaction mixture itself. Alternatively the deacetylation can also be carried out after the removal of the tertiary amide. The TGS thus obtained is then purified from the reaction mixture in various ways based on selective extraction into water immiscible solvent or solvents.

This prior art strategy had a problem. The selective and practically complete extraction of TGS into water immiscible solvent or solvents that have low miscibility in water is not very efficient. Since the solubility of TGS is very high in aqueous solutions, increased amount of solvents were required to be used for more repetitive extraction of TGS. This problem was sought to be removed in a process described in an earlier patent application WO2005/090374 A1, which describes a more practical and simpler process to obtain TGS in pure form, which is based on removal of dimethylformamide (DMF) prior to deacetylation of 6-acetyl-TGS, due to which more efficient extraction of 6-acetyl-TGS in organic solvents becomes possible, which can then be isolated free from the organic solvents, dissolved in water and deacylated. A highly efficient extraction into organic solvents was achieved when the product was still in 6-acetate form in aqueous solution. The inorganic and all polar impurities were left out in the aqueous solution and 6-acetyl-TGS was selectively extracted into the nearly water immiscible or water immiscible solvent such as ethyl acetate, butyl acetate, any other alkyl ester solvent, methyl tertiary butyl ether (MTBE), etc. The said organic solvent extract, was concentrated.

When this process is scaled up to the industrial level, the method described above gave rise to a problem of undesired formation of acid in case of use of ethyl acetate, butyl acetate and solvents containing ester group. The extract containing 6-acetyl-TGS in huge volumes in industrial reactors during concentration under long time intervals, when the solvent contained acetate or ester group, breaks down to acetic acid resulting in lowering of pH, which is highly detrimental to the product. When MTBE was used, problem of acetic acid formation did not occur. However, since the organic solvents containing ester group are also amongst the preferred solvents for the reasons of reasonable cost, availability and convenience considerations, it is important that the problem of acid formation caused by the solvents containing ester groups in high volume concentration be satisfactorily solved.

### SUMMARY OF THE INVENTION

It has been found that direct addition of solid acid neutralizing agents including addition of carbonates and bicarbonates is a very useful method of pH control in organic reactions when the reaction is predominantly in non-aqueous liquid containing medium and addition of water from external source is desired to be avoided as much as possible. The range of control of pH includes pH 6 to 8, preferably between 7 to 7.5. After the reaction is over, any excess of the solid neutralizing agent or other impurities formed can be very conveniently decanted or filtered off.

This invention was applied -more specifically to concentration of solutions or extracts of 6-acety-TGS or TGS in organic solvents containing ester group. It was surprising that such a simple method in organic synthesis production on industrial scale was never anticipated before.

This invention also covers use of organic solvents not having ester group for the purpose of extraction of 6-acetyl-TGS or TGS from reaction mixtures and concentrating them without the need to adjust the pH.

### BRIEF DESCRIPTION OF DRAWINGS AND SHORTFORMS

FIG. 1: Flow chart for the concentration of solvent extract of TGS & 6-acetyl TGS

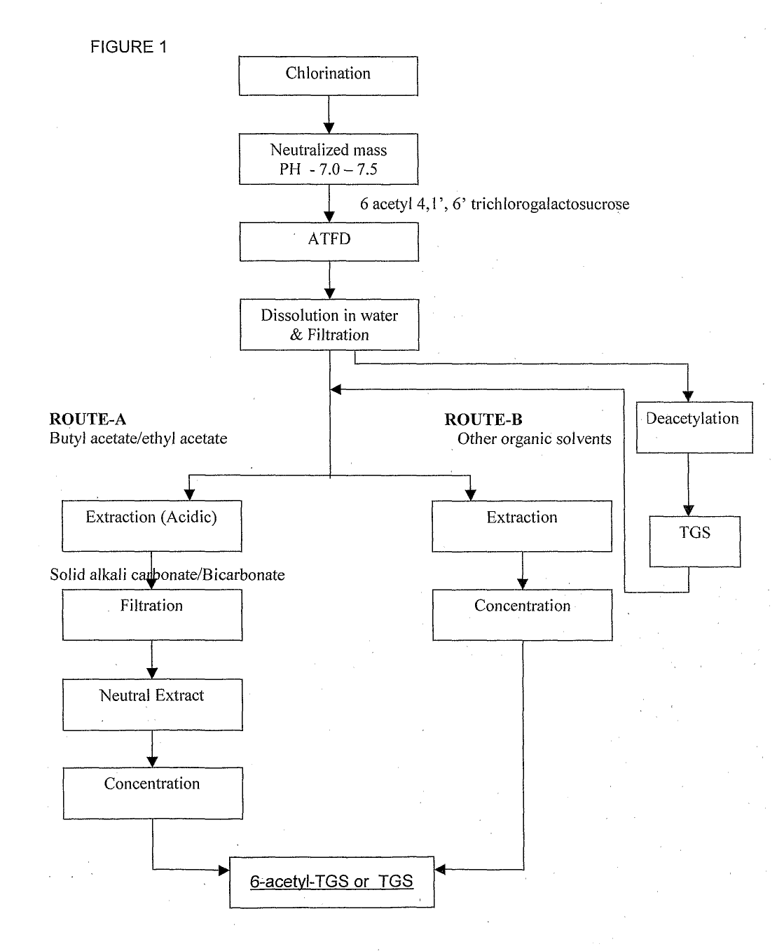

FIG. 2: Effect of concentration and addition of carbonates to the solvent extract on the 6-acetyl TGS content

TGS: 1′-6′-Dichloro-1′-6′-DIDEOXY-β-Fructofuranasyl-4-chloro-4-deoxy-galactopyranoside

i.e. 4,1′, 6′ trichlorogalactosucrose

6-Acetyl_TGS: 6-acetyl-4,1′, 6′trichlorogalactosucrose

HPLC: High Pressure Liquid Chromatography

TLC: Thin Layer Chromatography

### DETAILED DESCRIPTION OF INVENTION

In present invention, an improvement was introduced in this method whenever organic solvents having an ester group are required to be used, wherein acid neutralizing agents including carbonates and bicarbonates of any alkali metals such as sodium or potassium or any alkaline earth metals such as calcium or barium were added to maintain the pH neutral. However, use of the alkali for neutralization brought with it water associated with their solutions. This introduction of water in the reaction mixture was another detrimental factor. This problem could be avoided surprisingly by a very simple method, which was never anticipated in organic synthetic reactions at industrial scale manufacture, when these acid neutralizing compounds were directly added as solids and mixed in the reactor in solid form. After adjusting the pH, the solids were removed by filtration. The usage of aqueous medium was avoided.

When the solution to be concentrated is reaction mixture derived from any method of production of chlorination of sucrose, either containing TGS or 6-acetyl TGS, DMF and aqueous solvents were removed from it by any of the available methods, the remaining solids dissolved in preferred organic solvent containing ester group and subjected to concentration. The said ”available methods” in the instant case included ATFD drying as described in an earlier patent applications WO2005/090374 A1 and WO2005/090376 A1, and the solids recovered after drying were dissolved in preferred organic solvents containing ester group. It is anticipated here that it is always possible that methods of substantial removal, if not total removal, of DMF could be used including the method of steam stripping (Navia et al. 1996, U.S. Pat. No. 5,498,709) wherein such a low concentration of DMF is achieved that it results in acceptable reduction in interference caused by DMF in organic solvent extraction of 6-acetyl-TGS from the liquid reaction mixture; such a composition of liquid reaction mixture is also covered within the scope of application of this invention.

The syrup, remaining after concentration of the organic solvent extract under control of pH as described above (whenever organic solvents used is such as to lead to acidity production during concentration), is further purified by column chromatography and crystallized. Alternatively in the case of 6-acetylTGS, the same is deacetylated and TGS thus formed, which is already substantially free from impurities, is further purified by column chromatography.

The step of addition of carbonates or bicarbonates is not required if organic solvents selected for extraction in above step do not contain an ester group, thereby leaving no scope for the formation of an acid during prolonged distillation. One embodiment of this invention also covers use of such organic solvents (e.g. MTBE) not having an ester group for extraction of 6-acetyl-TGS from a reaction mixture and concentrating such an extract without the need of pH control.

The invention covers use of direct addition of carbonates and bicarbonates of any alkali metals such as sodium or potassium or any alkali earth metals such as calcium or barium, which were added to maintain the pH neutral during concentration of ester group containing organic solvents extract of 6-acetyl-TGS or TGS. The invention covers its application to compositions derived from any method of production other than the most preferred methods mentioned here for synthesis of 6-acetyl-TGS and TGS, including but not limiting to the use of enzymes, organo-tin catalysts, orthoesters, penta esters etc. The composition of matter to which pH control as described in this invention can be applied to include either a solution of or an extract of 6-acetyl-TGS in organic solvent or an organic solvent extract of TGS from its aqueous solution or a solution of TGS itself in organic solvents or as a process stream from a process of production of 6-acetyl-TGS or TGS. The said process of production of 6-acetyl-TGS or TGS includes, without being limited to, as described in Mufti et al. (1983) U.S. Pat. No. 4,380,476, Walkup et al. (1990 U.S. Pat. No. 4,980,463), Jenner et al. (1982) U.S. Pat. No. 4,362,869, Tulley et al. (1989) U.S. Pat. No. 4,801,700, Rathbone et al. (1989) U.S. Pat No. 4,826,962, Bornemann et al. (1992) U.S. Pat. No. 5,141,860, Navia et al. (1996) U.S. Pat. No. 5,498,709, Simpson (1989) U.S. Pat. No. 4,889,928, Navia (1990) U.S. Pat. No. 4,950,746, Neiditch et al. (1991) U.S. Pat. No. 5,023,329, Walkup et al. (1992) U.S. Pat. No. 5,089,608, Dordick et al. (1992) U.S. Pat. No. 5,128,248, Khan et al. (1995) U.S. Pat. No. 5,440,026, Palmer et al. (1995) U.S. Pat. No. 5,445,951, Sankey et al. (1995) U.S. Pat. No. 5,449,772, Sankey et al. (1995) U.S. Pat. No. 5,470,969, Navia et al. (1996) U.S. Pat. No. 5,498,709, Navia et al. (1996) U.S. Pat. No. 5,530,106 and patent applications containing similar patentable matter including in co-pending application Nos. WO 2005/090374 A1 and WO 2005/090376 A1.

The invention also covers use of solid carbonates and bicarbonates of any alkali metals such as sodium or potassium or any alkaline earth metals such as calcium or barium, to reactions involving organic solvents to control the pH or to maintain the pH neutral during any organic synthesis operations or processes.

Alternatively if the addition of carbonates/bicarbonates in solution form after dissolving the same in aqueous solutions is made, it resulted in formation of an aqueous layer in the ethyl acetate concentrate that requires an extra process stage of separating the layers. Furthermore the partitioning of product into the aqueous layer also occurs. Thus the control of pH by direct addition of solid carbonates/bicarbonates proved to be a highly efficient way of controlling the pH in organic solvent extract during concentration.

Described in the following are examples, which illustrate working of this invention without limiting the scope of this invention in any manner. Reactants, proportion of reactants used, range of reaction conditions are only illustrative and the scope extends to their analogous reactants, reaction conditions and reactions of analogous generic nature. This invention also covers organic reactions in general where drift of pH towards acidic side during the course of a non-aqueous extraction or the acidity present or developed for any reason is desired to be neutralized and pH raised to 7, around 7 or above without external addition of water with the pH adjusting agent.

Mention in singular is construed to cover its plural also viz: use of “an organic solvent” for extraction covers use of one or more organic solvents in succession or in combination.

### EXAMPLE 1

Reaction mixtures containing 6-acetyl-TGS are prepared by chlorination of sucrose derivative by Vilsmeier Haack reagent.

160 kg of sucrose was added to the reactor and was heated to 80° C. in DMF and 0.5 molar DBTO was added for the completion of the tin adduct. Acetylation was carried out with acetic anhydride. The 6-O-acetylation yield was 75% as reported by HPLC.

505 kg of PCl₅ was added to 1600 L of DMF taken in the glass lined reactor and the temperature was controlled below 30° C. The Vilsmeier-Haack reagent was allowed to form and the reactor contents was cooled to 0° C. with brine circulation. The 6-O-acetyl sucrose solution was added drop wise to the reaction mass and was allowed to attain room temperature.

The mixture was then heated to 80° C. and maintained for 3.0 hr. Then the mixture was further heated to 105° C. and maintained for 6 hours and again at 115° C. for 1 hour.

After chlorination, 2000 liters of the reaction mass is neutralized to pH 7.0-7.5.

The reaction mass is cooled to room temperature (25-30° C.) and centrifuged to remove suspended solids. The filtrate is passed through Agitated Thin Film Dryer (ATFD), to remove DMF. Details on ATFD are as per given in the patent applications WO2005/090374 A1 and WO2005/090376 A1. The solids obtained after ATFD were tested for DMF absence by gas chromatographic (GC) analysis.

The ATFD solids (800 kg) which contains 6-acetyl-TGS and other inorganic salts, were dissolved in 3-4 times w/v of water. The same could have been dissolved in any other volume range between 3 to 8 times W/V of water. The pH was adjusted to neutral and was filtered using appropriate filter aid to remove suspended solids. The presence of 6-acetyl-TGS in the solution was analyzed by TLC and HPLC.

The DMF free aqueous solution was extracted twice with 1:1 times of ethyl acetate. It could also be dissolved in other water immiscible solvent such as butyl acetate, any alkyl ester solvent twice. The organic layers were pooled together and concentrated. The Aqueous layer was analyzed for 6-acetyl-TGS content. The partitioning of 6-acetyl-TGS into the said organic layer was found to be highly efficient when compared to final Deacetylated hydroxy product. So more repetitive extractions or product loss in aqueous layer was avoided.

The organic layer was concentrated at 50-55° C. under vacuum. During the distillation of the extract in ethyl acetate or such other solvents, in large quantities (400 L and above), acetic acid is formed due to the breakage of ethyl acetate. The formation of acetic acid reduces the pH of the extract and causes product deterioration.

The breakdown of ethyl acetate to acetic acid when concentrated from 7500 L to 117 L, to acetic acid and the pH fall was recorded as follows as given in Table 1.

TABLE 1

| Ethyl acetate at | Acetic acid | pH of the |
|---|---|---|
| various stages | evaluation | ethyl |
| during | by GC | acetate |
| concentration (L) | (%) | solution |
| 7500 | 0 | 6.8 |
| 3750 | 0.2% | 6.8 |
| 1875 | 0.5% | 6.5 |
| 940 | 1.2% | 6.2 |
| 470 | 3.2% | 4.5 |
| 235 | 7.1% | 3.6 |
| 117 | 15.2% | 2.7 |

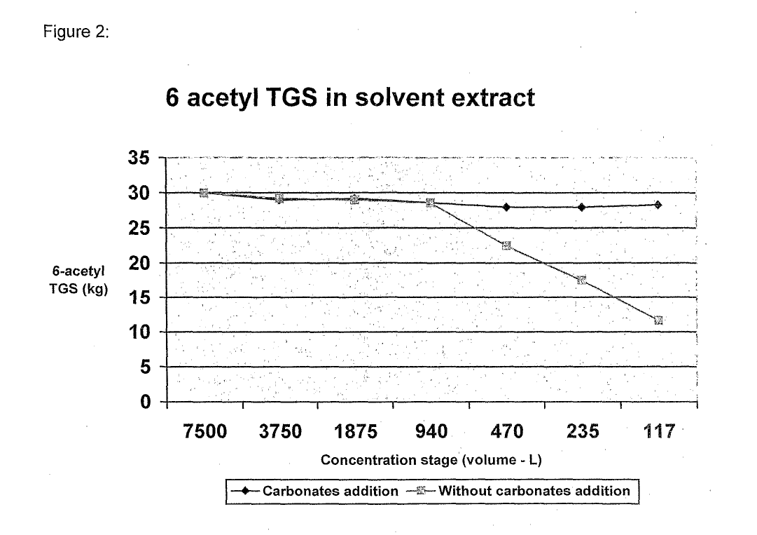

Addition of sodium carbonate was made to control the pH between 6.5-7.0. Quantity of 6-acetyl-TGS found in the concentrated mixture at various levels of concentration and with or without pH control was measured and is given in FIG. 2. It was seen that product present when a solution of 6-acetyl-TGS from 7500 liters to 117 liters was 28.3 kg, whereas when pH was not controlled, same was 11.2 kg. In both the cases, the content of the product when the concentration of the 7500 liters batch began was 30 kg. Thus it is clear that substantial losses occurred without carbonate addition and without pH control and the same can be significantly minimized by pH control. Considering high value of the product 6-acetyl-TGS and TGS, this improvement in process efficiency is very valuable.

After complete removal of organic layer by distillation, the syrup containing 6-acetyl-TGS is ready for further use.

### EXAMPLE 2

MTBE extraction of 6 acetyl TGS from aqueous mass was concentrated to remove the organic layer. The TGS loss was monitored at various stages.

Difference between concentration of 6-acetyl-TGS with and without addition of carbonates in ethyl acetate extract and content of the same during concentration of MTBE extract are given in Table 2.

TABLE 2

| | | 6 acetyl TGS | 6 acetyl TGS |
|---|---|---|---|
| Solvent extract | | content in Ethyl | content in Ethyl |
| at various stages | 6 acetyl TGS | acetate extract | acetate extract |
| during | content in | (without addn. | (with addn. Of |
| concentration (L) | MTBE extract | Of carbonates) | carbonate) |
| 3500 | 20.0 kg | 20.0 kg | 20.0 kg |
| 1750 | 20.0 kg | 19.8 kg | 20.0 kg |
| 875 | 19.9 kg | 18.54 kg | 19.8 kg |
| 435 | 19.92 kg | 16.52 kg | 19.82 kg |
| 215 | 19.65 kg | 11.42 kg | 19.6 kg |
| 100 | 19.6 kg | 9.26 kg | 19.65 kg |

The table shows clearly that in the solvent with ester group, such as in ethyl acetate, addition of the carbonates for the control of pH is a must to prevent product deterioration. Whereas the MTBE extract doesn't require any control of pH during concentration and the product deterioration is not seen.

### EXAMPLE 3

Reaction mixtures containing 6-acetyl-TGS are prepared by chlorination of sucrose derivative by Vilsmeier-Haack reagent by using methods as described in Example 1. After chlorination, 950 liters of the reaction mass is neutralized to pH 7.0-7.5.

The reaction mass is cooled to room temperature (25-30° C.) and centrifuged to remove suspended solids. The filtrate is passed through Agitated Thin Film Dryer (ATFD), to remove DMF. Details on ATFD are as per given in the patent applications WO2005/090374 A1 and WO2005/090376 A1. The solids obtained after ATFD were tested for DMF absence by gas chromatographic (GC) analysis.

The ATFD solids (400 kg) which contains 6-acetyl-TGS and other inorganic salts, were dissolved in 3-4 times w/v of water. The same could have been dissolved in any other volume range between 3 to 8. The pH was adjusted to 9.0 9.5 using calcium hydroxide slurry and deacetylation was monitored by TLC. After the deacetylation, the pH of the deacetylated mass was adjusted to neutral and filtered using appropriate filter aid to remove suspended solids.

The DMF free aqueous solution was extracted twice with 1:4 times ethyl acetate. It could also be dissolved in other water immiscible solvent such as butyl acetate, any alkyl ester solvent twice. The organic layers were pooled together and concentrated. The Aqueous layer was analyzed for TGS content. Complete extraction of TGS in the organic solvent was accomplished after 4 extractions.

The organic layer was concentrated at 50-55° C. under vacuum. During the distillation of the extract in ethyl acetate or such other solvents, in large quantities (400 L and above), acetic acid is formed due to the breakage of ethyl acetate. The formation of acetic acid reduces the pH of the extract and causes product deterioration.

The break down of ethyl acetate to acetic acid when concentrated from 6400 L to L, to acetic acid and the pH fall was recorded in Table 3 as follows.

TABLE 3

| Ethyl acetate at | Acetic acid | pH of the |
|---|---|---|
| various stages | evaluation | ethyl |
| during | by GC | acetate |
| concentration (L) | (%) | solution |
| 6400 | 0 | 6.6 |
| 3200 | 0.2% | 6.7 |
| 1600 | 0.4% | 6.5 |
| 800 | 0.8% | 5.8 |
| 400 | 2.6% | 3.9 |
| 200 | 6.8% | 3.2 |
| 100 | 14.5% | 2.3 |

This fall in pH was seen to be highly detrimental to the product during the concentration process. When pH was found below 6.0 any time during concentration, the addition of carbonates/bicarbonates helped in controlling the pH and was maintained between 6.5-7.0. Quantity of TGS found in the concentrated solvent at various levels of concentration and with or without pH control was measured. It was seen that product present when a solution of TGS was concentrated from 6400 liters to 100 liters was 27.6 kg when pH was controlled during concentration, whereas when pH was not controlled, same was 12.3 kg. In both the cases, the content of the product when the concentration of the 6400 liters batch began was 30 kg. Thus it is clear that substantial losses occurred without pH addition and the same can be significantly minimized by pH control. Considering high value of the product, this improvement in process efficiency is very valuable.

The product solution obtained after ethyl acetate concentration was filtered to remove the salts. The concentrated mass 100 kg was loaded on to 800 kg of silanized silica gel packed in chromatographic column. The elution was carried out with buffer solution at pH 9.0-9.5. The pure fractions were collected concentrated by reverse osmosis at room temperature, charcoalized and crystallized by suitable methods.

The pure TGS obtained was analyzed by HPLC and was found to be 98.73% and the overall yield was found to be 45%

1. A non-aqueous process of pH adjustment in large scale organic synthesis reactions comprising:
a. addition of acid neutralizing agents in solid form including solid carbonates and bicarbonates of alkali metals and alkaline earth metals to the reactants associated with a liquid medium
b. optionally removing solids present at the end of the process step by a process of separation of solids from liquids including decantation, filtration, ultrafiltration, centrifugation or any other means of solid liquid separation.

2. A process of claim 1 comprising its application to one or more process steps in a process of manufacture of chlorinated sucrose, their precursors or derivatives including 1′-6′-Dichloro-1′-6′-DIDEOXY-β-Fructofuranasyl-4-chloro-4-deoxy-galactopyranoside (TGS), 6-acetyl-1′-6′-Dichloro-1′-6′-DIDEOXY-β-Fructofuranasyl-4-chloro-4-deoxy-galactopyranoside, (6-acetyl-TGS).

3. A process of claim 2 wherein the said one or more process step comprises concentration of ester group containing organic solvent solution of one or more of:
a. a precursor or derivative of TGS including 6-ester-TGS, either alone or with other reactants, or
b. TGS alone or with other organic or inorganic reactant molecules.

4. A process of claim 3 wherein said ester containing organic solvent includes one or more of ethyl acetate, butyl acetate or any organic solvent having an ester group.

5. A process of claim 3, comprising pH adjustment within a range of pH 6 to 8, preferably within a range of pH 7 to 7.5.

6. A process of claim 5 wherein the said pH adjustment is done for a composition of reactants derived from in one or more of following ways:
a. dissolution of the TGS or 6-acetyl-TGS, in an aqueous medium
b. derived as a process stream from a process of production of TGS or 6-acetyl-TGS.

7. A process of extracting in MTBE or any solvent containing ketonic group, 6-acetyl-TGS or TGS from a composition of reactants of claim 6, concentrating the extract done in MTBE or the solvent containing ketonic group.
